SEQUENCE LISTING (10 sequences)

SEQ ID NO: 1 - DNA, 1248 bp, Escherichia coli
  gene (376)..(714): ytfP ORF
ggcgatgtcgcaacaagctgccttgtcttatttgctacgtggacaagggctggagagcgatcagagcgacagtgcggcaatgacctcgatgctgattggtttgggggttgcgcaaagtggccagattgtgggtaaaatcggcgagacgtttggcgtaagcaatttagcgctcgacacccagggagtaggcgactcctcccaggtagtggtcagcggctatgtattgccaggtctgcaagtgaaatacggcgtgggtatatttgactctatagcaacactcacgttacgttatcgcctgatgcctaagctatatctggaagccgtgtctggtgtagaccaggcactggatttgctctatcagttcgagttttagcaatgcgaatatttgtctacggcagtttacgccacaaacaaggcaacagtcactggatgaccaatgcccagttactgggcgatttcagtatcgataactaccagttgtatagcctgggccactatccaggcgcagttccggggaacggaacggtacacggtgaagtttatcgtattgacaacgccacgctggccgaacttgatgccttgcgcaccaggggcggtgaatacgcgcgccagttgattcagacgccgtacgggagtgcatggatgtacgtttatcaacgacccgtcgatggattaaagctaattgaaagcggcgactggttagacagggataagtaaccatatgcatacgccaccttcgggtggcgttgttttttgcgagacgactcgcattctgttttgtaattccctcaccttttgcttttctctccgagccgctttccatatctattaacgcataaaaaactctgctggcattcacaaatgcgcaggggtaaaacgtttcctgtagcaccgtgagttatactttgtataacttaaggaggtgcagatgcgtattaccataaaaagatgggggaacagtgcaggtatggtcattcccaatatcgtaatgaaagaacttaacttacagccggggcagagcgtggaggcgcaagtgagcaacaatcaactgattctgacacccatctccaggcgctactcgcttgatgaactgctggcacagtgtgacatgaacgccgcggaacttagcgagcaggatgtctggggtaaatccacccctgcgggtgacgaaatatggtaaagaaaagtgaatttgaacggggagacattgtgctggttggctttgatccagcaagcggccatgaacag

SEQ ID NO: 2 - DNA, 20 bp, Escherichia coli
  (1)..(20): ytfP-1
ggcgatgtcgcaacaagctg

SEQ ID NO: 3 - DNA, 20 bp, Escherichia coli
  (1)..(20): ytfP-2
ctgttcatggccgcttgctg

SEQ ID NO: 4 - DNA, 911 bp, Escherichia coli
  misc_feature (1)..(911): deletion-carrying ytfP-yjfA region
ggcgatgtcgcaacaagctgccttgtcttatttgctacgtggacaagggctggagagcgatcagagcgacagtgcggcaatgacctcgatgctgattggtttgggggttgcgcaaagtggccagattgtgggtaaaatcggcgagacgtttggcgtaagcaatttagcgctcgacacccagggagtaggcgactcctcccaggtagtggtcagcggctatgtattgccaggtctgcaagtgaaatacggcgtgggtatatttgactctatagcaacactcacgttacgttatcgcctgatgcctaagctatatctggaagccgtgtctggtgtagaccaggcactggatttgctctatcagttcgagttttagcaatgcgaattatgcatacgccaccttcgggtggcgttgttttttgcgagacgactcgcattctgttttgtaattccctcaccttttgcttttctctccgagccgctttccatatctattaacgcataaaaaactctgctggcattcacaaatgcgcaggggtaaaacgtttcctgtagcaccgtgagttatactttgtataacttaaggaggtgcagatgcgtattaccataaaaagatgggggaacagtgcaggtatggtcattcccaatatcgtaatgaaagaacttaacttacagccggggcagagcgtggaggcgcaagtgagcaacaatcaactgattctgacacccatctccaggcgctactcgcttgatgaactgctggcacagtgtgacatgaacgccgcggaacttagcgagcaggatgtctggggtaaatccacccctgcgggtgacgaaatatggtaaagaaaagtgaatttgaacggggagacattgtgctggttggctttgatccagcaagcggccatgaacag

SEQ ID NO: 5 - DNA, 21 bp, Escherichia coli
  (1)..(21): ilvGM1
caggacgaggaactaactatg

SEQ ID NO: 6 - DNA, 20 bp, Escherichia coli
  (1)..(20): ilvGM2
tcacgatggcggaatacaac

SEQ ID NO: 7 - DNA, 2111 bp, Escherichia coli
  RBS (8)..(12)
caggacgaggaactaactatgaatggcgcacagtgggtggtacatgcgttgcgggcacagggtgtgaacaccgttttcggttatccgggtggcgcaattatgccggtttacgatgcattgtatgacggcggcgtggagcacttgctgtgccgacatgagcagggtgcggcaatggcggctatcggttatgcccgtgctaccggcaaaactggcgtatgtatcgccacgtctggtccgggcgcaaccaacctgataaccgggcttgcggacgcactgttagattctatccctgttgttgccatcaccggtcaagtgtccgcaccgtttatcggcacggacgcatttcaggaagtggatgtcctgggattgtcgttagcctgtaccaagcacagctttctggtgcagtcgctggaagagttgccgcgcattatggctgaagcattcgacgttgccagctcaggtcgtcctggtccggttctggtcgatatcccaaaagatatccagctagccagcggtgacctggaaccgtggttcaccaccgttgaaaacgaagtgactttcccacatgccgaagttgagcaagcgcgccagatgctggcaaaagcgcaaaaaccgatgctgtacgttggtggtggcgtgggtatggcgcaggcagttcctgctttacgagaatttctcgctaccacaaaaatgcctgccacctgcacgctgaaagggctgggcgcagttgaagcagattatccgtactatctgggcatgctgggaatgcatggcaccaaagcggcgaacttcgcggtgcaggagtgcgacttgctgatcgccgtgggtgcacgttttgatgaccgggtgaccggcaaactgaacaccttcgcaccacacgccagtgttatccatatggatatcgacccggcagaaatgaacaagctgcgtcaggcacatgtggcattacaaggtgatttaaatgctctgttaccagcattacagcagccgttaaatatcaatgactggcagctacactgcgcgcagctgcgtgatgaacatgcctggcgttacgaccatcccggtgacgctatctacgcgccattgttgttaaaacaactgtcggatcgtaaacctgcggattgcgtcgtgaccacagatgtggggcagcaccagatgtgggccgcgcagcacatcgcacacactcgcccggaaaatttcattacctccagcggcttaggcaccatgggtttcggtttaccagcggcggttggcgcacaagtcgcacgaccgaacgatactgtcgtctgtatctccggtgacggctctttcatgatgaatgtgcaagagctgggcaccgtaaaacgcaagcagttaccgttgaaaatcgtcttactcgataaccaacggttagggatggttcgacaatggcagcaactgttttttcaggaacgatacagcgaaaccacccttactgataaccccgatttcctcatgttagccagcgccttcggcatccctggccaacacatcacccgtaaagaccaggttgaagcggcactcgacaccatgctgaacagtgatgggccatacctgcttcatgtctcaatcgacgaacttgagaacgtctggccgctggtgccgcctggcgccagtaattcagaaatgttggagaaattatcatgatgcaacatcaggtcaatgtatcggctcgcttcaatccggaaaccttagaacgtgttttacgcgtggtgcgtcatcgtggtttccacgtctgctcaatgaatatggctgccgccagcgatgcacaaaatataaatatcgaattgaccgttgccagcccacggtcggtcgacttactgtttagtcagttaaataaactggtggacgtcgcacacgttgccatctgccagagcacaaccacatcacaacaaatccgcgcctgagcgcaaaaggaatataaaaatgaccacgaagaaagctgattacatttggttcaatggggagatggttcgctgggaagacgcgaaggtgcatgtgatgtcgcacgcgctgcactatggcacctcggtttttgaaggcatccgttgctacgactcacacaaaggaccggttgtattccgccatcgtga

SEQ ID NO: 8 - protein, 548 aa, Escherichia coli
MNGAQWVVHALRAQGVNTVFGYPGGAIMPVYDALYDGGVEHLLCRHEQGAAMAAIGYARATGKTGVCIATSGPGATNLITGLADALLDSIPVVAITGQVSAPFIGTDAFQEVDVLGLSLACTKHSFLVQSLEELPRIMAEAFDVASSGRPGPVLVDIPKDIQLASGDLEPWFTTVENEVTFPHAEVEQARQMLAKAQKPMLYVGGGVGMAQAVPALREFLATTKMPATCTLKGLGAVEADYPYYLGMLGMHGTKAANFAVQECDLLIAVGARFDDRVTGKLNTFAPHASVIHMDIDPAEMNKLRQAHVALQGDLNALLPALQQPLNINDWQLHCAQLRDEHAWRYDHPGDAIYAPLLLKQLSDRKPADCVVTTDVGQHQMWAAQHIAHTRPENFITSSGLGTMGFGLPAAVGAQVARPNDTVVCISGDGSFMMNVQELGTVKRKQLPLKIVLLDNQRLGMVRQWQQLFFQERYSETTLTDNPDFLMLASAFGIPGQHITRKDQVEAALDTMLNSDGPYLLHVSIDELENVWPLVPPGASNSEMLEKLS

SEQ ID NO: 9 - DNA, 2111 bp, Escherichia coli; identical to SEQ ID NO: 7
  RBS (8)..(12)

SEQ ID NO: 10 - protein, 87 aa, Escherichia coli
MMQHQVNVSARFNPETLERVLRVVRHRGFHVCSMNMAAASDAQNINIELTVASPRSVDLLFSQLNKLVDVAHVAICQSTTTSQQIRA

(19) United States
(12) Patent Application Publication
(10) Pub. No.: US 2005/0089971 A1
(43) Pub. Date: Apr. 28, 2005

(54) Process for the preparation of d-pantothenic acid and/or its salts

(76) Inventor: Mechthild Rieping, Bielefeld (DE)

Correspondence Address: Fitch Even;Tabin & Flannery, Suite 401L, 1801 K Street NW, Washington, DC, 20006-1201, US

(21) Appl. No.: 10/490,851

(22) PCT Filed: Aug. 22, 2002
(86) PCT No.: PCT/EP02/09373

(30) Foreign Application Priority Data
Sep. 28, 2001 (DE) 101 47 960.3

(51) Int. Cl. C12P 13/04; C12N 15/74; C12N 1/21

(52) U.S. Cl. 435/106; 435/488; 435/252.33

(57) ABSTRACT

The invention provides a process for the preparation of D-pantothenic acid and/or its salts or animal feed additives containing them by the fermentation of microorganisms of the family Enterobacteriaceae, especially those which already produce D-pantothenic acid, wherein the nucleotide sequence(s) coding for the ytfP and/or yjfA open reading frames (ORF) are attenuated or, in particular, switched off, individually or together, in the microorganisms.

### FIELD OF THE INVENTION

The present invention relates to a process for the preparation of D-pantothenic-acid and/or its salts or mixtures containing them using microorganisms of the family Enterobacteriaceae in which at least the ytfP and/or yjfA open reading frame (ORF) is attenuated.

### PRIOR ART

Pantothenic acid is produced throughout the world on a scale of several thousand tons per year. It is used inter alia in human medicine, in the pharmaceutical industry and in the food industry. A large part of the pantothenic acid produced is used for feeding farm animals such as poultry and pigs.

Pantothenic acid can be prepared by chemical synthesis or biotechnologically by the fermentation of suitable microorganisms in suitable nutrient solutions. In the chemical synthesis, DL-pantolactone is an important precursor. It is prepared in a multistage process from formaldehyde, isobutyl aldehyde and cyanide. In subsequent process steps the racemic mixture is separated and the D-pantolactone is condensed with β-alanine to give D-pantothenic acid.

The typical commercial form is the calcium salt of D-pantothenic acid. The calcium salt of the racemic mixture of DL-pantothenic acid is also useful.

The advantage of preparation by the fermentation of microorganisms is the direct formation of the desired stereoisomeric form, namely the D form, free of L-pantothenic acid.

As indicated in EP-A 0 493 060, various species of bacteria, e.g. Escherichia coli (E. coli), Arthrobacter ureacfaciens, Corynebacterium erythrogenes and Brevibacterium ammoniagenes, as well as yeasts, e.g. Debaromyces castellii, can produce D-pantothenic acid in a nutrient solution containing glucose, DL-pantoic acid and β-alanine EP-A 0 493 060 further indicates that, in the case of E. coli, the formation of D-pantothenic acid is improved in a nutrient solution containing glucose, DL-pantoic acid and β-alanine by amplification of the pantothenic acid biosynthesis genes from E. coli which are contained on plasmids pFV3 and pFV5.

EP-A 0 590 857 and U.S. Pat. No. 5,518,906 describe mutants derived from the E. coli strain IFO03547, such as FV5714,FV525, FV814, FV521, FV221, FV6051 and FV5069, which carry resistances to various antimetabolites such as salicylic acid, α-ketobutyric acid, β-hydroxyaspartic acid, O-methylthreonine and α-ketoisovaleric acid. They produce pantoic acid in a nutrient solution containing glucose, and D-pantothenic acid in a nutrient solution containing glucose and β-alanine.- EP-A 0 590 857 and U.S. Pat. No. 5,518,906further indicate that, after amplification of the pantothenic acid biosynthesis genes panB, panC and panD, which are said to be contained on plasmid pFV31, in the above-mentioned strains, the production of D-pantoic acid is improved in nutrient solutions containing glucose and the production of D-pantothenic acid is improved in a nutrient solution containing glucose and β-alanine.

Furthermore, WO97/10340 reports the beneficial effect of amplification of the ilvGM operon on the production of D-pantothenic acid. Finally, EP-A 1 001 027 reports the effect of amplification of the panE gene on the formation of D-pantothenic acid.

The D-pantothenic acid or the corresponding salt is isolated from the fermentation broth and purified as in the prior art (EP-A 0 590 857 and WO 96/33283) and accordingly used in purified form, or the whole of the fermentation broth containing the D-pantothenic acid is dried (EP-A 1 050 219) and used in particular as an animal feed additive.

### OBJECT OF THE INVENTION

The inventors set out to provide novel measure to improve the preparation, by fermentation, of D-pantothenic acid and/or its salts or animal feed additives containing them.

### SUMMARY OF THE INVENTION

Whenever D-pantothenic acid, pantothenic acid or pantothenate is mentioned hereafter, this is understood as meaning not only the free acids but also the salts of D-pantothenic acid, e.g. the calcium, sodium, ammonium or potassium salt.

The invention provides a process for the preparation, by fermentation, of D-pantothenic acid and/or its salts or animal feed additives containing them by the fermentation of microorganisms of the family Enterobacteriaceae, especially those which already produce D-pantothenic acid, wherein
a) the nucleotide sequences coding for the ytfP and/or yifA open reading frames (ORF) are attenuated or, in particular, switched off under conditions suitable for reducing or switching off the intracellular activity of the appropriate protein (enzyme), optionally in combination with the attenuation or amplification of other genes, and
b) optionally in the presence of alkaline earth metal compounds introduced continuously or batchwise, preferably in stoichiometric amounts,
c) the D-pantothenic acid or the corresponding salt is enriched in the medium or the fermentation broth or in the cells of the microorganisms of the family Enterobacteriaceae, and
d) when the fermentation has finished, the D-pantothenic acid and/or the corresponding salt are isolated.

The invention also provides a process in which, when the fermentation has finished, all or part of the biomass (≧0 to 100%) is separated from the fermentation broth, or optionally remains in it, and the resulting broth is processed, optionally after concentration, to a solid mixture containing D-pantothenic acid and/or its salts together with other constituents of the fermentation broth.

The invention also provides microorganisms of the family Enterobacteriaceae, especially of the genus Escherichia, in which the ytfP ORF and/or yifA ORF or their gene products are present in attenuated form.

### DETAILED DESCRIPTION OF THE INVENTION

In this context the term ‘attenuation’ describes the reduction or switching-off, in a microorganism, of the intracellular activity of one or more enzymes or proteins coded for by the appropriate DNA, for example by using a weak promoter or using a gene or allele or an open reading frame (ORF) which codes for an appropriate enzyme or protein with a low activity or inactivates the appropriate gene, ORF or enzyme or protein, and optionally combining these measures.

An open reading frame (ORF) is a segment of a nucleotide sequence which codes or can code for a protein or polypeptide or a ribonucleic acid to which no function can be assigned according to the prior art. After a function has been assigned to the particular segment of the nucleotide sequence, the term ‘gene’ is generally used.

The attenuation measures, including the reduction of expression, generally reduce the activity or concentration of the appropriate protein to 0 to 75%, 0 to 50%, 0 to 25%, 0 to 10% or 0 to 5% of the activity or concentration of the wild-type protein or of the activity or concentration of the protein in the starting microorganism.

The microorganisms provided by the present invention can produce D-pantothenic acid from glucose, sucrose, lactose, fructose, maltose, molasses, starch and cellulose or from glycerol and ethanol. They are representatives of the Enterobacteriaceae, especially of the genus Escherichia. The species Escherichia coli may be mentioned in particular within the genus Escherichia. Suitable strains within the species Escherichia coli are the so-called K-12 strains, e.g. the strains MG1655 or W3110 (Neidhard et al.: Escherichia coli and Salmonella. Cellular and Molecular Biology (ASM Press, Washington D.C.)) or the Escherichia coli wild-type strain IFO3547 (Institute of Fermentation, Osaka, Japan) and mutants derived therefrom which are capable of producing D-pantothenic acid.

Examples of suitable D-pantothenic acid-producing strains of the genus Escherichia, especially of the species Escherichia coli, are:
Escherichia coli FV5069/pFV31
Escherichia coli FV5069/pFV202
Escherichia coli FE6/pFE80 and
Escherichia coli KE3

It has been found that the production of D-pantothenic acid by Enterobacteriaceae is improved after attenuation of the ytfP and/or yjfA open reading frames.

The nucleotide sequence of the ytfP ORF is published under Accession Number AAC77179 and the nucleotide sequence of the yjfA ORF is published under Accession Number AAC77180 at the National Center for Biotechnology Information (NCBI, Bethesda, Md., USA). They can also be taken from the genome sequence of Escherichia coli published by Blattner et al. (Science 277, 1453-1462 (1997)).

The open reading frames described in the cited literature references can be used according to the invention. It is also possible to use a alleles which result from the degeneracy of the genetic code or from neutral sense mutations.

Attenuation can be achieved for example by reducing or switching off the expression of the ytfP ORF and/or yjfA ORF or the catalytic properties of the protein. Both measures may optionally be combined.

Reduction of the gene expression can be achieved by an appropriate culture technique, by genetic modification (mutation) of the signal structures of gene expression or by antisense RNA technology. Examples of signal structures of gene expression are repressor genes, activator genes, operators, promoters, attenuators, ribosome binding sites, the start codon and terminators. Those skilled in the art can find relevant details inter alia in e.g. Jensen and Hammer (Biotechnology and Bioengineering 58: 191-195 (1998)), Carrier and Keasling (Biotechnology Progress 15, 58-64 (1999)), Franch and Gerdes (Current Opinion in Microbiology 3, 159-164 (2000)) and well-known textbooks on genetics and molecular biology, for example the textbook by Knippers (“Molekulare Genetik”, 6 th edition, Georg Thieme Verlag, Stuttgart, Germany, 1995) or the textbook by Winnacker (“From Genes to Clones”, VCH Verlagsgesellschaft, Weinheim, Germany, 1990).

Mutations which lead to a change or reduction of the catalytic properties of enzyme proteins are known from the prior art. Examples which may be mentioned are the studies by Qiu and Goodman (Journal of Biological Chemistry 272: 8611-8617 (1997)), Yano et al. (Proceedings of the National Academy of Sciences, USA 95, 5511-5515 (1998)) and Wente and Schachmann (Journal of Biological Chemistry 266, 20833-20839 (1991)). Surveys can be found in well-known textbooks on genetics and molecular biology, e.g. the textbook by Hagemann (“Allgemeine Genetik”, Gustav Fischer Verlag, Stuttgart, 1986).

Possible mutations are transitions, transversions, insertions and deletions. Depending on the effect of amino acid exchange on enzyme activity, the term ‘missense mutations’ or ‘nonsense mutations’ is used. Insertions or deletions of at least one base pair in a gene lead to frame shift mutations, the effect of which is to incorporate false amino acids or terminate the translation prematurely. Deletions of several codons typically result in a complete disappearance of enzyme activity. Instructions on the production of such mutations belong to the prior art and can be found in well-known textbooks on genetics and molecular biology, e.g. the textbook by Knippers (“Molekulare Genetik”, 6 th edition, Georg Thieme Verlag, Stuttgart, Germany, 1995), the textbook by Winnacker (“From Genes to Clones”, VCH Verlagsgesellschaft, Weinheim, Germany, 1990) or the textbook by Hagemann (“Allgemeine Genetik”, Gustav Fischer Verlag,Stuttgart, 1986)

Suitable mutations in the ytfP ORF and/or yjfA ORF, for example deletion mutations, can be incorporated into suitable strains by gene or allele exchange.

A common method is that of gene exchange using a conditionally replicating pSC101 derivative, pMAK705,as described by Hamilton et al. (Journal of Bacteriology 171, 4617-4622 (1989)). Other methods described in the prior art, for example that of Martinez-Morales et al. (Journal of Bacteriology 181, 7143-7148 (1999)) or that of Boyd et al. (Journal of Bacteriology 182, 842-847(2000)), can likewise be used.

Also, mutations in the ytfP ORF and/or yjfA ORF or mutations which affect the expression of the ytfP ORF and/or yjfA ORF can be transferred to different strains by conjugation or transduction.

Furthermore, for the production of D-pantothenic acid with strains of the family Enterobacteriaceae, it can be advantageous not only to attenuate the ytfP ORF and/or yjfA ORF, but also to amplify or, in particular, overexpress one or more endogenous genes, i.e. genes inherent in the particular species, selected from the group comprising:
the ilvGM operon coding for acetohydroxy acid synthase II (WO97/10340),
the panB gene coding for ketopantoate hydroxymethyltansferase (U.S. Pat. No. 5,518,906),
the panE gene coding for ketopantoate reductase (EP-A-1 001 027),
the panD gene coding for aspartate decarboxylase (U.S. Pat. No. 5,518,906),
the panC gene coding for pantothenate synthetase (U.S. Pat. No. 5,518,906),
the serC gene coding for phosphoserine transaminase (Duncan and Coggins, Biochemical Journal 234: 49:-57 (1986)),
the gcvT, gcvH and gcvP genes coding for the glycine cleavage system (Okamura-Ikeda et al., European Journal of Biochemistry 216, 539-548 (1993)), and
the glyA gene coding for serine hydroxymethyltransferase (Plamann et al. (Nucleic Acids Research 11(7): 2065-2075 (1983))).

In this context the term ‘amplification’ describes the increase, in a microorganism, of the intracellular activity of one or more enzymes or proteins coded for by the appropriate DNA, for example by increasing the copy number of the gene or genes, using a strong promoter or a gene or allele coding for an appropriate enzyme or protein with a high activity, and optionally combining these measures.

Through the measures of amplification, especially overexpression, the activity or concentration of the appropriate protein is generally increased at least by 10%, 25%, 50%, 75%, 100%, 150%, 200%, 300%, 400% or 500%, and at most by up to 1000% or 2000%, based on that of the wild-type protein or the activity or concentration of the protein in the starting microorganism.

Finally, for the production of D-pantothenic acid with strains of the family Enterobacteriaceae, it can be advantageous not only to attenuate the ytfP ORF and/or yjfA ORF, but also to attenuate or, in particular, switch off or express at a low level the following genes, individually or together:
the avta gene coding for transaminase C (EP-A-1 001 027),
the poxB gene coding for pyruvate oxidase (Grabau and Cronan, Nucleic Acids Research 14:(13), 5449-5460 (1986)), and
the pckA gene coding for PEP carboxykinase (Medina et al., Journal of Bacteriology 172, 7151-7156 (1990)).

Furthermore, for the production of D-pantothenic acid, it can be advantageous not only to attenuate the ytfP ORF and/or yjfA ORF, but also to switch off unwanted secondary reactions (Nakayama: “Breeding of Amino Acid Producing Microorganisms”, in: Overproduction of Microbial Products, Krurphanzl, Sikyta, Vanek (eds.), Academic Press, London, UK, 1982). In the process according to the invention, it is possible to use bacteria in which the metabolic pathways which reduce the formation of D-pantothenic acid are at least partially switched off.

The microorganisms prepared according to the invention can be cultivated by the batch process, the fed batch process or the repeated fed batch process. A summary of known cultivation methods is provided in the textbook by Chmiel (Bioprozesstechnik 1. Einführung in die Bioverfahrenstechnik (Gustav Fischher Verlag, Stuttgart, 1991)) or in the textbook by Storhas (Bioreaktoren und periphere Einrichtungen (Vieweg Verlag, Brunswick/Wiesbaden, 1994)).

The culture medium to be used must appropriately meet the demands of the particular strains. Descriptions of culture media for various microorganisms can be found in “Manual of Methods for General Bacteriology” of the American Society for Bacteriology (Washington D.C., USA, 1981). Carbon sources which can be used are sugars and carbohydrates, e.g. glucose, sucrose, lactose, fructose, maltose, molasses, starch and cellulose, oils and fats, e.g. soya bean oil, sunflower oil, groundnut oil and coconut fat, fatty acids, e.g. palmitic acid, stearic acid and linoleic acid, alcohols, e.g. glycerol and ethanol, and organic acids, e.g. acetic acid. These substances can be used individually or as a mixture.

Nitrogen sources which can be used are organic nitrogen-containing compounds such as peptones, yeast extract, meat extract, malt extract, corn steep liquor, soya bean flour and urea, or inorganic compounds such as ammonium sulfate, ammonium chloride, ammonium phosphate, ammonium carbonate and ammonium nitrate. The nitrogen sources can be used individually or as a mixture.

Phosphorus sources which can be used are phosphoric acid, potassium dihydrogenphosphate or dipotassium hydrogenphosphate or the corresponding sodium salts. The culture medium must also contain metal salts, e.g. magnesium sulfate or iron sulfate, which are necessary for growth. Finally, essential growth-promoting substances such as amino acids and vitamins can be used in addition to the substances mentioned above. D-pantothenic acid precursors, such as aspartate, β-alanine, ketoisovalerate, ketopantoic acid or pantoic acid, and optionally their salts, can also be added to the culture medium. Said feed materials can be added to the culture all at once or fed in appropriately during cultivation.

The pH of the culture is controlled by the appropriate use of basic compounds such as sodium hydroxide, potassium hydroxide, ammonia or aqueous ammonia, or acid compounds such as phosphoric acid or sulfuric acid.

As a further possibility, to prepare the alkaline earth metals salts of pantothenic acid, especially the calcium salt, a suspension or solution of an inorganic compound containing an alkaline earth metal, for example calcium hydroxide, or of an organic compound such as an alkaline earth metal salt of an organic acid, for example calcium acetate, is added continuously or batchwise during the fermentation. In this way the cation required to prepare the desired alkaline earth metal salt of D-pantothenic acid is introduced into the fermentation broth directly in the desired amount generally in a proportion of 0.8 to 1.2, based on the pantothenic acid, and preferably in stoichiometric amounts.

Foaming can be controlled using antifoams such as fatty acid polyglycol esters. The stability of plasmids can be maintained by adding suitable-selectively acting substances, e.g. antibiotics, to the medium. Aerobic conditions are maintained by introducing oxygen or oxygen-containing gaseous mixtures, .e.g. air, into the culture. The temperature of the culture is normally 25° C. to 45° C. and preferably 30° C. to 40° C. The culture is continued until the formation of D-pantothenic acid has reached a maximum. This objective is normally achieved within 10 hours to 160 hours.

The D-pantothenic acid or corresponding D-pantothenic acid salts contained in the fermentation broth can then be isolated and purified according to the prior art.

It is also possible to remove all or part (≧0 to 100%) of the biomass from the fermentation broths containing D-pantothenic acid and/or its salts, preferably initially by means of known methods of separation, for example. centrifugation, filtration, decantation or a combination thereof. Another possibility, however, is to leave the whole of the biomass in the fermentation broth.

In general the suspension or solution is preferably concentrated and processed to a powder, for example using a spray dryer or a freeze-drying unit. This powder is then generally converted by means of suitable compaction or granulation processes, e.g. pelletizing, to a coarser, easily flowable, storable and largely dust-free product with the desired particle size distribution of optionally 20 to 2000 μm, especially of 100 to 1400 μm. In the granulation or compaction process it is advantageous to use conventional organic or inorganic auxiliary substances or carriers like starch, gelatin, cellulose derivatives or similar substances, such as those conventionally used as binders, gelling agents or thickeners in food or animal feed processing, or to use other substances like silicic acids, silicates or stearates.

Alternatively, the fermentation product, with or without other conventional constituents of the fermentation broth, can be absorbed onto an organic or inorganic carrier known and conventionally used in animal feed processing, for example silicic acids, silicates, meals, brans, flours, starches, sugars or the like, and/or stabilized with conventional thickeners or binders. Relevant application examples and processes are described in the literature (Die Mühle+Mischfuttertechnik 132 (1995) 49, page 817).

Optionally, at a suitable stage of the process, D-pantothenic acid, the desired D-pantothenic acid salt or a preparation containing these compounds is added in order to attain the desired content of pantothenic acid or the desired salt, or adjust said content to the desired value, in the end product.

The desired content generally ranges from 20 to 80 wt. % (dry weight).

The concentration of pantothenic acid can be determined by known chemical methods (Velisek; Chromatographic Science 60, 515-560 (1992)) or microbiological methods, e.g. the Lactobacillus plantarum test (DIFCO MANUAL, 10 th edition, pp 1100-1102;Michigan, USA).

A pure culture of the Escherichia coli K-12 strain DH5α/pMAK705 was deposited as DSM 13720 on 08 Sep. 2000 in the Deutsche Sammlung für Mikroorganismen und Zelliulturen (DSMZ, Brunswick, Germany) under the terms of the Budapest Treaty.

A pure culture of the Escherichia coli K-12 strain FE6-1 was deposited as DSM 13721 on 08 Sep. 2000 in the Deutsche Sammlung für Mikroorganismen und Zellkulturen (DSMZ, Brunswick, Germany) under the terms of the Budapest Treaty.

The present invention is illustrated in greater detail below with the aid of embodiment examples.

The minimum medium (M9) and complete medium (LB) used for Escherichia coli are described by J. H. Miller (A Short Course In Bacterial Genetics (1992), Cold Spring Harbor Laboratory Press). The isolation of plasmid DNA from Escherichia coli, as well as all the techniques for restriction, ligation, Klenow treatment and alkaline phosphatase treatment, are performed according to Sambrook et al. (Molecular Cloning—A Laboratory Manual (1989) Cold Spring Harbor Laboratory Press). Unless described otherwise, the transformation of Escherichia coli is performed according to Chung et al. (Proceedings of the National Academy of Sciences of the United States of America, USA (1989) 86: 2172-2175).

The incubation temperature in the preparation of strains and transformants is 37° C. Temperatures of 30° C. and 44° C. are used in the gene exchange process according to Hamilton et al.

### EXAMPLE 1

Construction of the Deletion Mutation of the ytfP-yjfA Gene Region

The ytfP-yjfA gene region is amplified from Escherichia coli K12 using the polymerase chain reaction (PCR) and synthetic oligonucleotides. The following PCR primers (MWG Biotech, Ebersberg, Germany) are synthesized from the nucleotide sequence of the ytfP-yjfA gene region in E. coli K12.MG1655 (SEQ ID No. 1):

(SEQ ID No. 2)

ytfP-1: 5′ - GGCGATGTCGCAACAAGCTG - 3′

(SEQ ID No. 3)

ytfP-2: 5′ - CTGTTCATGGCCGCTTGCTG - 3′

The chromosomal E. coli K12 MG1655 DNA used for the PCR is isolated with “Qiagen Genomic-tips 100/G” (QIAGEN, Hilden, Germany) according to the manufacturer's instructions. An approx. 1250 bp DNA fragment can be amplified with the specific primers under standard PCR conditions (Innis et al. (1990) PCR Protocols. A Guide to Methods and Applications, Academic Press) using Taq DNA polymerase (Gibco-BRL, Eggenstein, Germany). The PCR product is ligated with vector pCR2.1TOPO (TOPO TA Cloning Kit, Invitrogen, Groningen, The Netherlands) according to the manufacturer's instructions and transformed into the E. coli strain TOP10F′. Plasmid-carrying cells are selected on LB agar to which 50 μg/ml of ampicillin have been added. After isolation of the plasmid DNA, the successful cloning of the PCR product is checked with the restriction enzymes EcoRI and NsiI.

To produce a 337 bp deletion in the ytfP-yjfA region, vector pCR2.1TOPOytfP-yjfA is cleaved with the restriction enzymes NdeI and SspI and the 4.8 kbp DNA fragment is treated with the Klenow enzyme and then ligated.

The E. coli strain DH5α is transformed with the ligation mixture and plasmid-carrying cells are selected on LB agar to which 50 μg/ml of ampicillin have been added. After isolation of the plasmid DNA, plasmids in which the mutagenic DNA sequence shown in SEQ ID No. 4 is present in cloned form are detected by control cleavage with the enzyme EcoRI. The plasmid is called pCR2.1TOPOΔjyfA.

### EXAMPLE 2

Construction of Exchange Vector pMAK705ΔyjfA

The ytfP-yjfA allele described in Example 1 is isolated from vector pCR2.1TOPOΔyjfA after restriction with the enzymes SacI and XbaI and separation in 0.8% agarose gel, and ligated with plasmid pMAK705 (Hamilton et al. (1989) Journal of Bacteriology 171, 4617 4622) digested with the enzymes SacI and XbaI. The ligation mixture is transformed into DH5α and plasmid-carrying cells are selected on LB agar to which 20 μg/ml of chloramphenicol have been added. The successful cloning is detected after isolation of the plasmid DNA and cleavage with the enzymes SacI and XbaI. The exchange vector formed, pMAK705ΔyjfA (=pMAK705deltayjfA), is shown in FIG 1.

### EXAMPLE 3

Site-Specific Mutagenesis of the ytfP-yjfA Gene Region in the E. coli Strain FE6-1

The E. coli strain FE6 is a valine-resistant mutant of E. coli K12 MG1655 (US-B-6171845) and is deposited as DSM12379 in the Deutsche Sammlung für Mikroorganismen und Zellkulturen (DSMZ, Brunswick, Germany). Spontaneous mutants are isolated from FE6 after incubation at 37° C. on minimal agar to which 2 g/l of glucose and 1 g/l of β-hydroxyaspartic acid have been added.

A chosen β-hydroxyaspartic acid-resistant single colony is then incubated at 37° C. on minimal agar containing 2 g/l of glucose and 0.2 g/l of O-methylthreonine. After this step, a mutant called FE6-1 is resistant to valine, α-ketoisovalericacid, β-hydroxyaspartic acid and O-methylthreonine. For exchange of the chromosomal ytfP-yjfA gene region with the plasmid-coded deletion construct, FE6-1 is transformed with plasmid pMAK705ΔyjfA. The gene exchange is effected by the selection method described by Hamilton et al. (1989) Journal of Bacteriology 171, 4617-4622, and is verified by standard PCR methods (Innis et al. (1990) PCR Protocols. A Guide to Methods and Applications, Academic Press) using the following oligonucleotide primers:

(SEQ ID No. 2)

ytfP-1: 5′ - GGCGATGTCGCAACAAGCTG - 3′

(SEQ ID No. 3)

ytfP-2: 5′ - CTGTTCATGGCCGCTTGCTG - 3′

The strain obtained is called FE6-1ΔyjfA.

### EXAMPLE 4

Preparation of D-Pantothenic Acid with the Strain FE6-1ΔyjfA/pFV31ilvGM

4.1 Amplification and Cloning of the ilvGM Gene

The ilvGM operon from Escherichia coli IFO3547 coding for acetohydroxy acid synthase II (Institute of Fermentation, Osaka, Japan) is amplified using the polymerase chain reaction (PCR) and synthetic oligonucleotides. PCR primers (MWG Biotech, Ebersberg, Germany) are synthesized from the nucleotide sequence of the ilvGM operon in E. coli K12 MG1655 (GenBank: Accession No. M87049). The sequence of the ilvGM1 primer is chosen so that the primer contains an adenine in position 8. This generates a modified ribosome binding site 7 nucleotides upstream from the start codon of the ilvG proteins.

(SEQ ID No. 5)

ilvGM1: 5′ - CAGGACGAGGAACTAACTATG - 3′

(SEQ ID NO. 6)

ilvGM2: 5′ - TCACGATGGCGGAATACAAC - 3′

The chromosomal E. coli IFO3547 DNA used for the PCR is isolated with “QIAGEN Genomic-tips 100/G” (QIAGEN, Hilden, Germany) according to the manufacturer's instructions. An approx. 2100 bp DNA fragment comprising the modified ribosome binding site, the ilvGM coding regions and approx. 180 bp 3′-flanking sequences can be amplified with the specific primers under standard PCR conditions (Innis et. al.: PCR Protocols. A Guide to Methods and Applications, 1990, Academic Press) using Pfu DNA polymerase (Promega Corporation, Madison, USA). The PCR product is cloned into plasmid pCR-BluntII-TOPO and transformed into the E. coli strain TOP10 (Invitrogen, Groningen, The Netherlands, product description Zero Blunt TOPO. PCR Cloning Kit, Cat. No. K2800-20).

The successful cloning is detected by cleavage of plasmid pCR-BluntIFO3547ilvGM with the restriction enzymes EcoRI and SphI. To do this, the plasmid DNA is isolated by means of the “QIAprep Spin Plasmid Kit” (QIAGEN, Hilden, Germany), cleaved and then separated in a 0.8% agarose gel. The DNA sequence of the amplified fragment is determined using the reverse and universal sequencing primers (QIAGEN, Hilden, Germany). The sequence of the PCR product is shown in SEQ ID No. 7 and 9. The ilvG gene or allele is identified in SEQ ID No. 7. The ilvM gene or allele is identified in SEQ ID No. 9. The corresponding gene products or proteins are shown in SEQ ID No. 8and 10.

4.2 Cloning of the ilvGM Genes into Expression Vector pTrc99A

For expression in Escherichia coli K12, the ilvGM genes described in Example 4.1. are cloned into vector pTrc99A (Amersham Pharmacia Biotech Inc., Uppsala, Sweden). This is done by cleaving plasmid pCR-BluntIFO3547ilvGM with the enzyme EcoRI, separating the cleavage mixture in 0.8%. agarose gel and isolating the 2.1.kbp ilvGM fragment using the QIAquick Gel Extraction Kit (QIAGEN, Hilden, Germany). Vector pTrc99A is cleaved with the enzyme EcoRI, an alkaline phosphatase treatment is carried out and the product is ligated with the isolated ilvGM fragment. The ligation mixture is transformed into the E. coli strain DH5α. pTrc99A-carrying cells are selected on LB agar (Lennox, Virology 1: 190 (1955)) to which 50 μg/ml of ampicillin have been added. The successful cloning of the ilvGM operon can be detected after isolation of the plasmid DNA and control cleavage with SalI and SphI. In the vector called pTrc99AilvGM (FIG. 2), expression of the ilvGM operon is regulated by the Ptrc promoter located upstream from the modified ribosome binding site and by the rRNA terminator region located downstream from the ilvGM coding regions.

4.3 Construction of Vector pFV31ilvGM

The D-pantothenic acid-producing E. coli strain FV5069/pFV31 is described in EP-A-0 590 857 and is deposited as FERM BP 4395 under the terms of the Budapest Treaty. Plasmid pFV31 is isolated from FV5069/pFV31 and cleaved with the enzyme BamHI and the protruding 3′ ends are treated with the Klenow enzyme This is followed by an alkaline phosphatase treatment. After restriction with the enzyme SspI and separation of the cleavage mixture in 0.8% agarose gel, the 2.8 kbp ilvGM expression cassette is isolated from vector pTrc99AilvGM described in Example 4.2 and ligated with linearized and dephosphorylated vector pFV31. The ligation mixture is transformed into the E. coli strain DH5α and plasmid-carrying cells are selected on LB agar to which 50 μg/ml of ampicillin have been added. The successful cloning of the ilvGM expression cassette can be detected after isolation of the plasmid DNA and control cleavage with HindIII, SalI, SmaI, SphI and XbaI. The plasmid is called pFV31ilvGM (FIG. 3).

4.4 Preparation of the Strain FE6-lΔyjfA/pFV31ilvGM

The strain FE6-1ΔyjfA obtained in Example 3 and the strain FE6-1 are transformed with plasmid pFV31ilvGM and transformants are selected on LB agar supplemented with 50 μg/ml of ampicillin. The strains FE6-1ΔyjfA/pFV31ilvGM and FE6-1/pFV31ilvGM are formed in this way.

4.5 Preparation of D-Pantothenic Acid with the Strain FE6-1ΔyjfA/pFV31ilvGM

The pantothenate production of the E. coli strains FE6-1/PFV31ilvGM and FE6-1ΔyjfA/pFV31ilvGM is checked in batch cultures of 10 ml contained in 100 ml Erlenmeyer flasks. To do this, 10 ml of a preculture medium of the following composition: 2 g/l of yeast extract, 10 g/l of (NH₄)₂SO₄, 1 g/l of KH₂PO₄, 0.5 g/l of MgSO₄*7H₂O, 15 g/l of CaCO₃, 20 g/l of glucose and 50 μg/ml of ampicillin, are inoculated with a single colony and incubated for 20 hours at 33° C. and 200 rpm on an ESR incubator from Kühner AG (Birsfelden, Switzerland). 200 μl aliquots of this preculture are inoculated into 10 ml of production medium (25 g/l of (NH₄)₂SO₄, 2 g/l of KH₂PO₄, 1 g/l of MgSO₄*7H₂O, 0.03 g/l of FeSO₄*7H₂O, 0.018 g/l of MnSO₄·1H₂O, 30 g/l of CaCO₃, 20 g/l of glucose, 20 g/l of β-alanine, 250 mg/l of thiamine) and incubated for 48 hours at 37° C. and 200 rpm. After incubation, the optical density (OD) of the culture suspension is determined with an LP2W photometer from Dr. Lange (Düsseldorf, Germany) at a wavelength of 660 nm.

The concentration of D-pantothenate formed is then determined in the sterile-filtered culture supernatant by means of the Lactobacillus plantarum ATCC8014 pantothenate assay according to DIFCO's instructions (DIFCO MANUAL, 10 th edition, pp 1100-1102; Michigan, USA). Calibration is effected using calcium D(+)-pantothenate hydrate (catalogue number 25, 972-1, Sigma-Aldrich, Deisenhofen, Germany).

The experimental results are shown in Table 1.

TABLE 1

| | OD | Pantothenate |
|---|---|---|
| Strain | (660 nm) | g/l |
| FE6-1/pFV31ilvGM | 11.1 | 0.78 |
| FE6-1ΔyjfA/ | 10.0 | 1.02 |
| pFV31ilvGM | | |

### BRIEF DESCRIPTION OF THE FIGURES

FIG. 1: pMAK705ΔyjfA (=pMAK705deltayjfA)

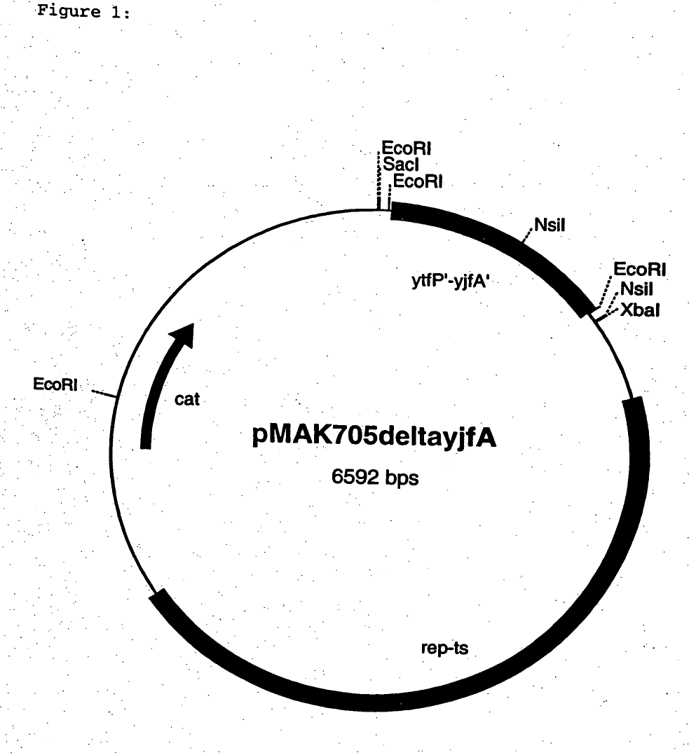

FIG. 2: pTrc99AilvGM

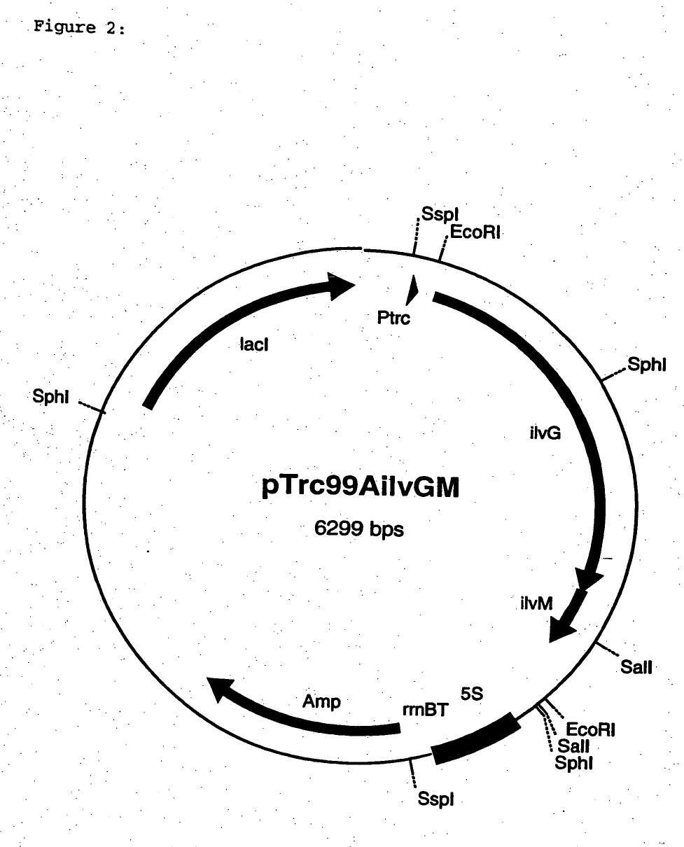

FIG. 3: pFV31ilvGM

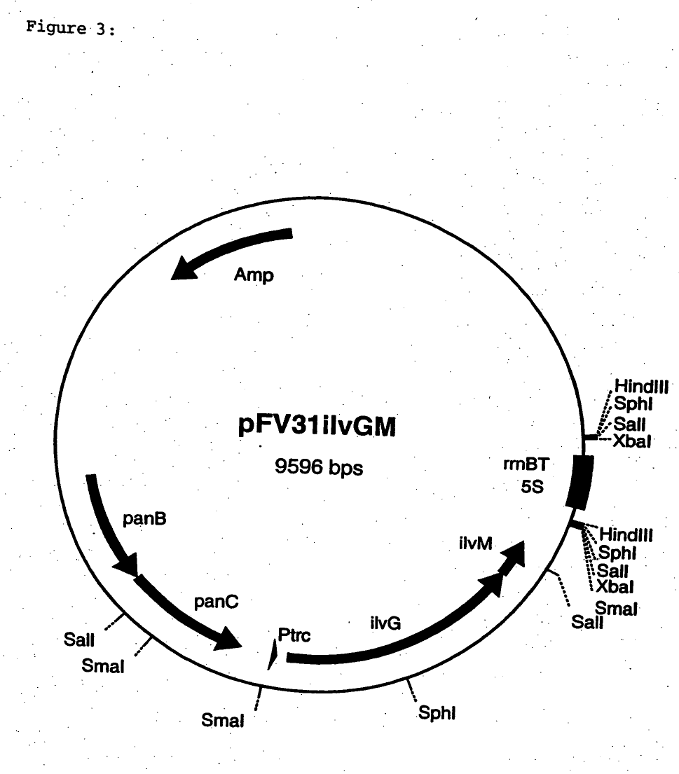

Length data are to be understood as approximate. The abbreviations and symbols used have the following meanings:
cat: chloramphenicol resistance gene
rep-ts: temperature-sensitive replication region of plasmid pSC101
ytfP′-yjfA′: DNA sequence containing truncated coding regions of ytfP and yjfA
Amp: ampicillin resistance gene
lacI: gene for the repressor protein of the trc promoter
Ptrc: trc promoter region, IPTG-inducible
ilvG: coding region of the large subunit of acetohydroxy acid synthase II
ilvM: coding region of the small subunit of acetohydroxy acid synthase II
5S: rRNA region
rrnBT: rRNA terminator region
panB: coding region of the panB gene
panC: coding region of the panC gene

The abbreviations for the restriction enzymes have the following meanings:
EcoRI: restriction endonuclease from Escherichia coli
HindIII restriction endonuclease from Haemophilus influenzae
NsiI: restriction endonuclease from Neisseria sicca
SacI: restriction endonuclease from Streptomyces achromogenes
SalI: restriction endonuclease from Streptomyces albus
SmaI: restriction endonuclease from Serratia marcescens
SphI: restriction endonuclease from Streptomyces phaeochromogenes
SspI: restriction endonuclease from Sphaerotilus species
XbaI: restriction endonuclease from Xanthomonas badrii

1-16. (canceled)

17. A microorganism of the family Enterobacteriaceae comprising at least one nucleotide mutation resulting in the attenuation of either the ytfP or yifA open reading frame product of said microorganism, and wherein said microorganism makes D-pantothenic acid.

18. The microorganism of claim 17, wherein at least one endogenous gene product, other than those produced by the ytfP or yifA open reading frame, is either attenuated or amplified.

19. The microorganism of claim 18, wherein at least one endogenous gene product, other than that produced by the ytfP or yifA open reading frame, is amplified by the overexpression of one or more genes selected from the group consisting of:
a) the ilvGM operon coding for acetohydroxy acid synthase II;
b) the panB gene coding for ketopantoate hydroxymethyltransferase;
c) the panE gene coding for ketopantoate reductase;
d) the panD gene coding for aspartate decarboxylase;
e) the panC gene coding for pantothenate synthetase;
f) the serC gene coding for phosphoserine transaminase;
g) the gcvT, gcvH and gcvP genes coding for the glysine cleavage system; and
h) the glyA gene coding for serine hydroxymethyltransferase.

20. The microorganism of claim 18, wherein a metabolic pathway that reduces the formation of D-pantothenic acid is at least partially switched off.

21. The microorganism of claim 18, wherein at least one endogenous gene product, other than that produced by the ytfP or yifA gene open reading frame, is attenuated by reducing the expression of one or more genes selected from the group consisting of:
a) the avtA gene coding for transaminase C;
b) the poxB gene coding for pyruvate oxidase; and
c) the pckA gene coding for PEP carboxykinase.

22. The microorganism of claim 18, wherein said nucleotide mutation is a transition, transversion, insertion or deletion.

23. The microorganism of any one of claim 17-22, wherein said microorganism is of the genus Escherichia.

24. The microorganism of claim 23, wherein said microorganism is of the species Escherichia coli.

25. A process for the preparation of D-pantothenic acid or a salt of D-pantothenic acid, comprising:
a) fermenting the microorganism of any one of claims 17-22 to produce a fermentation broth;
b) enriching either said microorganism or said fermentation broth in said D-pantothenic acid or salt of D-pantothenic acid; and
c) collecting either said microorganism or said fermentation broth after the enrichment of step b).

26. The process of claim 25, further comprising isolating said D-pantothenic acid or salt of D-pantothenic acid from the enriched microorganism or enriched fermentation broth collected in step c) by removing biomass or other constituents present.

27. The process of claim 26, wherein the amount of said D-pantothenic acid or salt of D-pantothenic acid is adjusted in said fermentation broth so that, upon drying, said D-pantothenic acid or salt of D-pantothenic acid, constitutes 20-80 weight % (dry weight) of the product.

28. The process of claim 27, wherein said D-pantothenic acid or salt of D-pantothenic acid is in the form of a magnesium or calcium salt.

29. The process of claim 25, wherein said process is used to produce an alkaline earth metal salt of D-pantothenic acid.

30. The process of claim 25, wherein said fermenting is carried out in the presence of one or more alkaline metal salts that are introduced during fermentation either continuously or in a batchwise manner and wherein an alkali metal salt of D-pantothenic acid is recovered from the enriched fermentation broth of step c).

31. The process of claim 26, further comprising concentrating said D-pantothenic acid or salt of said D-pantothenic acid after it has been isolated.

32. A process for the preparation of an animal feed additive, comprising:
converting the D-pantothenic acid or salt of D-pantothenic acid produced by the process of claim 25 into a dry, flowable composition having a particle size distribution of 20-2000 μm.

33. The process of claim 32, wherein said particle size distribution is 100-1400 μm.

34. The process of claim 32, wherein said D-pantothenic acid or salt of D pantothenic acid is a magnesium or calcium salt.

35. The process of claim 34, wherein the concentration of said magnesium or calcium salt of D-pantothenic acid in said animal feed additive is adjusted so that it constitutes 20 to 80 wt. % (dry weight).

36. The process of claim 32, wherein said animal feed additive is in a pulverulent or granular form.

37. The process of claim 36, wherein said pulverulent or granular form of said animal feed additive is produced from said fermentation broth by a procedure selected from the group consisting of:
a) drying and compaction;
b) spray drying;
c) spray drying and granulation; and
d) spray drying and pelleting.
